(19) United States
(12) Patent Application Publication
(10) Pub. No.: US 2025/0160502 A1
(43) Pub. Date: May 22, 2025

(54) STICK-SHAPED COSMETIC AND METHOD FOR PRODUCING STICK-SHAPED COSMETIC

(71) Applicant: TOKIWA CORPORATION, Gifu (JP)

(72) Inventors: Minoru IKEDA, Saitama (JP); Toshio MAEDA, Gifu (JP)

(73) Assignee: TOKIWA CORPORATION, Gifu (JP)

(21) Appl. No.: 18/953,683

(22) Filed: Nov. 20, 2024

(30) Foreign Application Priority Data
Nov. 22, 2023 (JP) 2023-198611
Nov. 22, 2023 (JP) 2023-198615

(51) Int. Cl. A45D 40/20; A61Q 1/02

(52) U.S. Cl. CPC A45D 40/20; A61Q 1/02

(57) ABSTRACT

A stick-shaped cosmetic has a cross section perpendicular to an axial direction having a multicolor region including a plurality of regions color-coded with N types of colors (N is an integer of 2 or more). The cross section includes a first cross section and a second cross section different from the first cross section. An area ratio of the regions of each color in the multicolor region of the first cross section is equal to an area ratio of the regions of each color in the multicolor region of the second cross section.

### TECHNICAL FIELD

The present disclosure relates to a stick-shaped cosmetic and a method for producing a stick-shaped cosmetic.

### BACKGROUND

For example, as a stick-shaped cosmetic such as an eyebrow pencil and an eye shadow pencil, a stick-shaped cosmetic of which a cross section perpendicular to an axial direction has a multicolor region including a plurality of color-coded regions is known. As this type of technology, for example, Japanese Unexamined Patent Publication No. H11-29439 discloses a pencil type or mechanical pencil type cosmetic having a core body (stick-shaped cosmetic) having several types of elongated and divided color streaks, and a method for producing the same.

### SUMMARY

In the stick-shaped cosmetic as described above, fineness and randomness can be imparted to a color pattern of appearance thereof, and aesthetics can be enhanced, but since it is not possible to achieve consistent color usage at the time of use, there is room for improvement in usability.

Therefore, an object of one aspect of the present disclosure is to provide a stick-shaped cosmetic excellent in aesthetics and usability.

A stick-shaped cosmetic according to one aspect of the present disclosure is (1) a stick-shaped cosmetic of which a cross section perpendicular to an axial direction has a multicolor region including a plurality of regions color-coded with N types of colors (N is an integer of 2 or more), in which the cross section includes a first cross section and a second cross section different from the first cross section, and an area ratio of the regions of each color in the multicolor region of the first cross section is equal to an area ratio of the regions of each color in the multicolor region of the second cross section.

In this stick-shaped cosmetic, aesthetics can be enhanced by a color pattern of the multicolor region of the cross section. In addition, the area ratio of the regions of each color in the multicolor region of the first cross section is equal to the area ratio of the regions of each color in the multicolor region of the second cross section. Therefore, it is possible to express a same application color (that is, consistent color usage) in each of a case of applying so that respective colors of the multicolor region are mixed when the first cross section is an application surface and a case of applying so that respective colors of the multicolor region are mixed when the second cross section is an application surface, and it is possible to obtain practical usability. Therefore, according to the present disclosure, it is possible to obtain a stick-shaped cosmetic having excellent aesthetics and usability.

(2) In the above (1), the multicolor region may include a pattern composed of at least one of a dot pattern, a marble pattern, a camouflage pattern, and a mosaic pattern. In this case, aesthetics can be further enhanced by the color pattern of the multicolor region.

(3) In the above (1) or (2), an arrangement of the regions of the respective colors in the multicolor region of the first cross section may be different from an arrangement of the regions of the respective colors in the multicolor region of the second cross section. In this case, different patterns can be included in the multicolor region of the first cross section and the multicolor region of the second cross section.

(4) In any one of the above (1) to (3), a side surface of the stick-shaped cosmetic may include a stripe pattern composed of a plurality of linear regions extending along the axial direction and color-coded. In this case, aesthetics can be further enhanced by the stripe pattern on the side surface.

(5) In any one of the above (1) to (4), among the plurality of regions color-coded with the N types of colors in the multicolor region, an area of a widest maximum region may be 30% or less of a total area of the multicolor region, and an area of a narrowest minimum region may be 0.1% or more of the total area of the multicolor region. In this case, a fine color pattern can be included in the multicolor region.

(6) In any one of the above (1) to (5), a number of times that a center line passing through a center of the multicolor region intersects boundary lines of the plurality of regions color-coded with the N types of colors may be five times or more. In this case, a fine color pattern can be included in the multicolor region.

(7) In any one of the above (1) to (6), boundary lines of the plurality of regions color-coded with the N types of colors in the multicolor region may be amorphous. In this case, a color pattern composed of a plurality of regions having an amorphous outline can be included in the multicolor region.

(8) In any one of the above (1) to (7), a predetermined color obtained by mixing the N types of colors may be expressed as an application color. In this case, when the stick-shaped cosmetic is applied so that the respective colors in the multicolor region are mixed, the predetermined color can be expressed as the application color.

In a method for producing the stick-shaped cosmetic as described above, kneaded grains of the respective colors are kneaded so as not to be mixed with each other to form an intermediate body (multicolor ball), and the intermediate body is extruded by a molding machine to produce the stick-shaped cosmetic. Therefore, for example, in the vicinity of an extrusion port (nozzle) of the molding machine, the intermediate body may be rubbed against an inner wall surface of the molding machine and stretched in an extrusion direction. In this case, the side surface of the produced stick-shaped cosmetic may become dirty due to mixing of the plurality of types of colors, and aesthetics of the stick-shaped cosmetic may be impaired.

Therefore, another aspect of the present disclosure is to provide a method for producing a stick-shaped cosmetic capable of enhancing aesthetics.

A method for producing a stick-shaped cosmetic according to another aspect of the present disclosure is (9) a method for producing a stick-shaped cosmetic of which a cross section perpendicular to an axial direction has a multicolor region including a plurality of regions color-coded with N types of colors (N is an integer of 2 or more), the method including: a first step of preparing a plurality of monochromatic core materials configuring the stick-shaped cosmetic for each of the N types of colors; and a second step of, after the first step, extruding the plurality of the core materials of the N types of colors with the axial direction aligned or an intermediate body including the plurality of the core materials of the N types of colors along the axial direction by a molding machine to obtain the stick-shaped cosmetic.

In this method for producing a stick-shaped cosmetic, when the intermediate body is extruded in the axial direction by the molding machine, even when the intermediate body is rubbed against the inner wall surface of the molding machine along the axial direction, the core materials included in the intermediate body are stretched in the axial direction, and it is possible to suppress mixing of the plurality of different core materials, and thus it is possible to suppress mixing of the plurality of types of colors. Therefore, in the produced stick-shaped cosmetic, it is possible to suppress the side surface thereof from becoming dirty and to enhance aesthetics.

(10) In the above (9), in the second step, the plurality of core materials of the N types of colors in the intermediate body may be randomly arranged. In this case, a color pattern of the multicolor region can be randomly configured.

(11) In the above (9) or (10), in the second step, the plurality of core materials of the N types of colors in the intermediate body may be arranged so as to form a pattern composed of at least one of a dot pattern, a marble pattern, a camouflage pattern, and a mosaic pattern when viewed from the axial direction. In this case, in the produced stick-shaped cosmetic, aesthetics can be enhanced by the color pattern included in the multicolor region.

(12) In any one of the above (9) to (11), the second step may include a step of bundling the plurality of core materials of the N types of colors while mixing them with the axial direction aligned, and packing the plurality of core materials into a cylindrical member, and a step of forming a columnar intermediate body by compressing and integrating the plurality of core materials packed in the cylindrical member along the axial direction. In this case, molding by the molding machine can be easily and reliably performed.

(13) In any one of the above (9) to (12), a cross-sectional shape of the core material may be a polygonal shape. In this case, a core material having a polygonal cross-sectional shape can be used.

(14) In any one of the above (9) to (13), a maximum width of the cross section of the core material may be 0.1 to 10 mm. In this case, a fine color pattern can be included in the multicolor region of the produced stick-shaped cosmetic.

(15) In any one of the above (9) to (14), an oily component of the core material may be 30 mass % or more and 80 mass % or less. In this case, since the intermediate body is easily stretched when rubbed against the inner wall surface of the molding machine, the above effect of suppressing mixing of the plurality of types of colors is particularly effective.

(16) In any one of the above (9) to (15), the intermediate body may include 12 or more core materials. In this case, various color patterns can be included in the multicolor region of the produced stick-shaped cosmetic.

### BRIEF DESCRIPTION OF THE DRAWINGS

FIG. 1 is a perspective view illustrating a cosmetic device including a stick-shaped cosmetic according to an embodiment;

FIG. 2 is a side view illustrating the stick-shaped cosmetic of FIG. 1;

FIG. 3A is a photograph corresponding to a cross section taken along line A1-A1 in FIG. 2; FIG. 3B is a cross-sectional view corresponding to a cross section taken along line A2-A2 in FIG. 2;

FIG. 4 is a cross-sectional view corresponding to a cross section taken along line A3-A3 in FIG. 2;

FIG. 5 is a flowchart illustrating a method for producing the stick-shaped cosmetic according to the embodiment;

FIG. 6 is a perspective view illustrating an example of an intermediate body;

FIG. 7 is a view for describing the method for producing the stick-shaped cosmetic according to the embodiment;

FIG. 8 is a view for describing continuation of FIG. 7;

FIG. 9 is a photograph illustrating a cross section of a stick-shaped cosmetic according to a first modification;

FIG. 10 is a photograph illustrating a cross section of a stick-shaped cosmetic according to a second modification; and

FIG. 11 is a photograph illustrating a cross section of a stick-shaped cosmetic according to a third modification.

### DETAILED DESCRIPTION

Hereinafter, an embodiment according to the present disclosure will be described with reference to the drawings. In the description of the drawings, the same or corresponding elements are denoted by the same reference numerals, and redundant description is appropriately omitted. Some of the drawings may be simplified or exaggerated for easy understanding, and dimensional ratios and the like are not limited to those described in the drawings.

In the present disclosure, a stick-shaped cosmetic is, for example, a rouge, a lipstick, a lip liner, a lip gloss, an eyeliner, an eyebrow-paint, an eyeshadow, a concealer, or a cosmetic stick. The stick-shaped cosmetic may be a stick-shaped product including a material that is solid at normal temperature (for example, 25° C.), or may be a stick-shaped product including a flexible material (for example, a semi-solid form, a soft-solid form, a jelly form, a mousse form, a paste containing these, or the like) in place of or in addition to this.

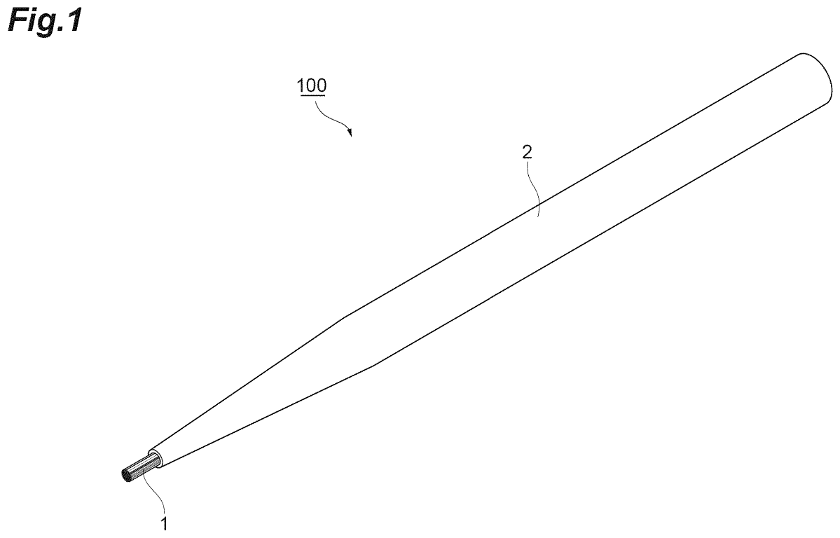

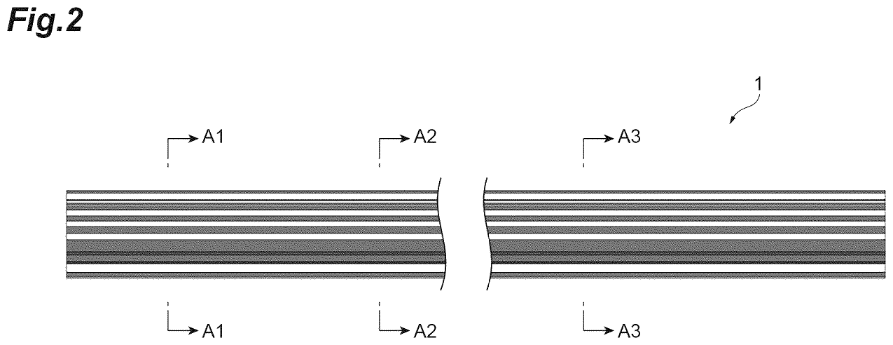

As illustrated in FIGS. 1 and 2, a stick-shaped cosmetic 1 has a stick shape having a circular cross section. The stick-shaped cosmetic 1 is accommodated in a tapered cylindrical container 2 to configure a cosmetic device 100. The cosmetic device 100 can be used by feeding the stick-shaped cosmetic 1 from a tip side of the container 2 by a feeding mechanism (not illustrated). That is, in the cosmetic device 100, the stick-shaped cosmetic 1 can be applied in a state where a part of the appearance thereof is exposed. Here, the stick-shaped cosmetic 1 is applied in a state where an end surface of the stick-shaped cosmetic 1 in an axial direction (hereinafter, also simply referred to as “axial direction”) and at least a part of an outer peripheral surface (side surface) are exposed.

In the cosmetic device 100, the stick-shaped cosmetic 1 may be loaded on a wooden shaft or a resin shaft, or may be loaded on a feeding cartridge. The stick-shaped cosmetic 1 may be an oily stick-shaped cosmetic, and for example, an oily component may be 30 mass % or more and 80 mass % or less. The stick-shaped cosmetic 1 may include a pearl pigment. The cross-sectional shape of the stick-shaped cosmetic 1 is not particularly limited, and may be a polygonal shape, an elliptical shape, a flat circular shape, a droplet shape (also referred to as a teardrop shape), or the like.

The outer peripheral surface of the stick-shaped cosmetic 1 includes a stripe pattern. Here, the entire outer peripheral surface of the stick-shaped cosmetic 1 has a stripe pattern. The stripe pattern on the outer peripheral surface of the stick-shaped cosmetic 1 extends along the axial direction and is composed of a plurality of linear regions color-coded with N types of colors (N is an integer of 2 or more). Widths of the respective stripes of the stripe pattern (dimension in a circumferential direction of each of the plurality of linear regions) may be different from each other, or widths of some stripes may be the same. A color arrangement pattern of the stripe pattern (color arrangement of each stripe) is randomly set.

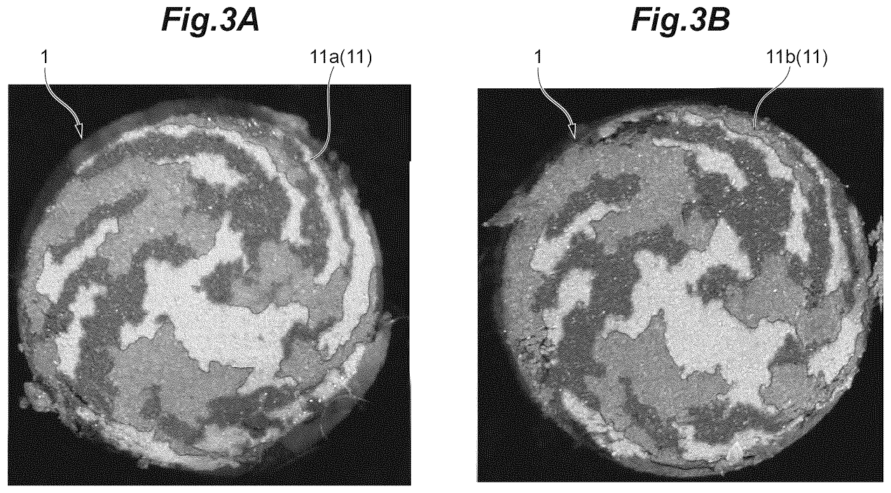

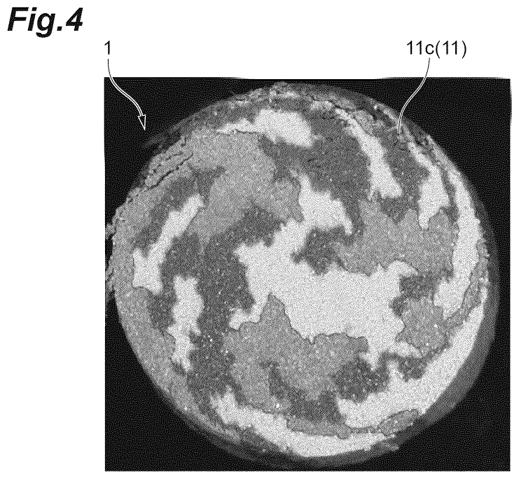

As illustrated in FIGS. 3A, 3B, and 4, the stick-shaped cosmetic 1 has a cross section perpendicular to the axial direction having a multicolor region 11 including a plurality of regions color-coded with the N types of colors. Here, the multicolor region 11 is a region appearing in the entire region of the cross section perpendicular to the axial direction in the stick-shaped cosmetic 1. The multicolor region 11 is a region appearing in the cross section at any position in the axial direction in the stick-shaped cosmetic 1. The multicolor region 11 includes a pattern composed of at least one of a dot pattern, a marble pattern, a camouflage pattern, and a mosaic pattern.

The dot pattern is, for example, a pattern in which a plurality of dots (balls) are regularly or irregularly arranged. The marble pattern is, for example, a pattern imitating marble, and is a pattern in which spirals or streaks are finely superimposed or flow-like shapes are superimposed in a plurality of colors. The camouflage pattern is, for example, a pattern that gives the impression of blending into the forest or the like, and is also referred to as a camouflage pattern. The mosaic pattern is, for example, a pattern formed by gathering small pieces.

An area of a widest maximum region among the plurality of regions color-coded with the N types of colors in the multicolor region 11 is 30% or less of a total area of the multicolor region 11. An area of a narrowest minimum region among the plurality of regions color-coded with the N types of colors in the multicolor region 11 is 0.10% or more of the total area of the multicolor region 11. Boundary lines of the plurality of regions color-coded with the N types of colors in the multicolor region 11 are amorphous. The boundary lines of the plurality of regions color-coded with the N types of colors in the multicolor region 11 are not constant straight lines, are not constant curves, and extend with fluctuation or disturbance. A number of times that a center line passing through a center of the multicolor region 11 intersects the boundary lines of the plurality of regions color-coded with the N types of colors is five times or more, preferably ten times or more, more preferably 15 times or more, and still more preferably 20 times or more.

An area of any of the plurality of regions color-coded with the N types of colors in the multicolor region 11 may be 0.1% to 30% or 0.2% to 5% of the entire multicolor region 11.

There may be 30 or more regions of each color in the multicolor region 11. Here, the multicolor region 11 includes approximately 30 A color regions, approximately 30 B color regions, and approximately 30 C color regions.

The stick-shaped cosmetic 1 can express a predetermined color obtained by mixing the N types of colors in the multicolor region 11 as an application color. That is, the stick-shaped cosmetic 1 can express the predetermined color as the application color by mixing respective colors in the multicolor region 11 by amounts corresponding to an area ratio of the regions of each color in the multicolor region 11. As an example, in the stick-shaped cosmetic 1, the area ratio of the A color region, the B color region, and the C color region of the multicolor region 11 is 1:1:1, and the predetermined color obtained by mixing the A color, the B color, and the C color at 1:1:1 is set as the application color. The predetermined color is theoretically the same color as the mixture of three colors of A, B, and C. For example, the A color may be blue, the B color may be orange, the C color may be green, and the predetermined color may be brown.

In the present embodiment, a multicolor region 11a (FIG. 3A) of a first cross section at an arbitrary position of the stick-shaped cosmetic 1, a multicolor region 11b (FIG. 3B) of a second cross section at an arbitrary position different from the first cross section, and a multicolor region 11c (FIG. 4) of a third cross section at an arbitrary position different from the first cross section and the second cross section have the following relationship.

That is, the area ratio of the regions of each color in the multicolor region 11a included in the first cross section is equal to the area ratio of the regions of each color in the multicolor region 11b included in the second cross section. In other words, a ratio of a total area of the A color region, a total area of the B color region, and a total area of the C color region in the multicolor region 11a is equal to a ratio of a total area of the A color region, a total area of the B color region, and a total area of the C color region in the multicolor region 11b. The area ratio of the regions of each color in the multicolor region 11b included in the second cross section is equal to the area ratio of the regions of each color in the multicolor region 11c included in the third cross section. In other words, the ratio of the total area of the A color region, the total area of the B color region, and the total area of the C color region in the multicolor region 11b is equal to a ratio of a total area of the A color region, a total area of the B color region, and a total area of the C color region in the multicolor region 11c.

An arrangement of the regions of the respective colors in the multicolor region 11a included in the first cross section is different from an arrangement of the regions of the respective colors in the multicolor region 11b included in the second cross section. In other words, a shape of a pattern of the multicolor region 11a and/or a color arrangement pattern of the pattern are different from a shape of a pattern of the multicolor region 11b and/or a color arrangement pattern of the pattern. An arrangement of the regions of the respective colors in the multicolor region 11b included in the second cross section is different from an arrangement of the regions of the respective colors in the multicolor region 11c included in the third cross section. In other words, the shape of the pattern of the multicolor region 11b and/or the color arrangement pattern of the pattern are different from a shape of a pattern of the multicolor region 11c and/or a color arrangement pattern of the pattern.

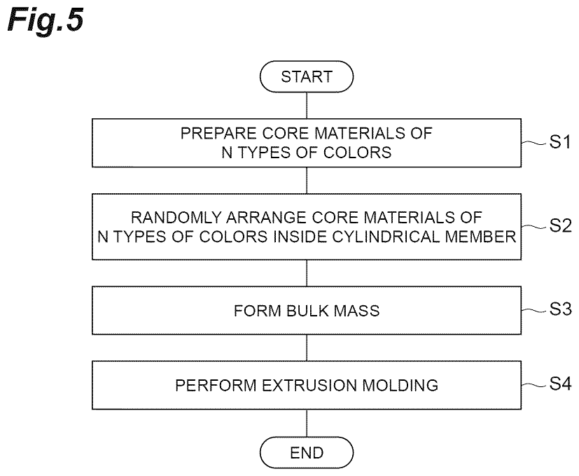

Next, a method for producing the stick-shaped cosmetic 1 will be described with reference to a flowchart of FIG. 5.

First, a plurality of monochromatic core materials configuring the stick-shaped cosmetic 1 are prepared for each of N types of colors (step S1: first step). The core material is a thin stick-shaped material, and a maximum width of the cross section of a core material is 0.1 to 10 mm. A cross-sectional shape of the core material is not particularly limited, and is, for example, a circular shape. The cross section of the core material may have a polygonal shape as described later. An oily component of the core material is 30 mass % or more and 80 mass % or less. The core material may include a pearl pigment. 12 or more core materials are used, and for example, 20 core materials are used for each of the N types of colors, and a total of 60 core materials are used. For example, a width of the core material is a largest value among dimensions passing through a center in the cross section orthogonal to the axial direction.

Subsequently, a cylindrical member having a bottomed cylindrical shape is prepared, and the plurality of core materials of the N types of colors are packed inside the cylindrical member with the axial direction aligned along the axial direction of the cylindrical member (step S2). In step S2 described above, the plurality of core materials of the N types of colors are bundled while being mixed with the axial direction aligned, and are packed into the cylindrical member without any gap. Thus, the plurality of core materials of the N types of colors are randomly arranged inside the cylindrical member without any gap. Here, the plurality of core materials of the N types of colors are arranged such that a bulk mass 51 obtained in the subsequent step forms a pattern composed of at least one of a dot pattern, a marble pattern, a camouflage pattern, and a mosaic pattern when viewed from the axial direction.

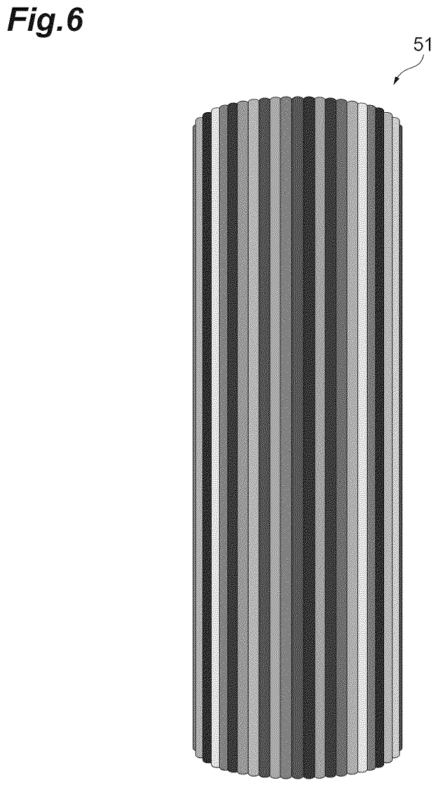

Subsequently, the plurality of core materials packed inside the cylindrical member are compressed and integrated in the axial direction by using a press machine. Thereby, a cylindrical bulk mass (intermediate body) 51 illustrated in FIG. 6 is formed (step S3). The bulk mass 51 includes the plurality of core materials of the N types of colors. In the bulk mass 51, the plurality of core materials are integrated. An outer peripheral surface of the bulk mass 51 has a stripe pattern composed of a plurality of regions color-coded with N types of colors. The stripe pattern on the outer peripheral surface of the bulk mass 51 only needs to be along the axial direction, and may have a linear shape, a dogleg shape, a zigzag shape, or a curved shape.

The dogleg shape is a shape in which a straight line extends while being bent once along the axial direction. Here, the dogleg shape is a shape that extends linearly from one side to the other side in the axial direction, is then bent, and extends linearly. The zigzag shape is a shape extending in a zigzag shape along the axial direction. In other words, the zigzag shape is a shape in which a straight line extends while being bent a plurality of times along the axial direction. Here, the zigzag shape is a shape in which a straight line extends linearly from one side to the other side in the axial direction, then bends, and this is repeated a plurality of times. Widths of the respective stripes of the stripe pattern may be different from each other according to the arrangement of the plurality of core materials at an outer edge portion inside the cylindrical member, or the widths of some stripes may be the same. The color arrangement pattern of the stripe pattern is randomly set according to the arrangement of the plurality of core materials at the outer edge portion inside the cylindrical member.

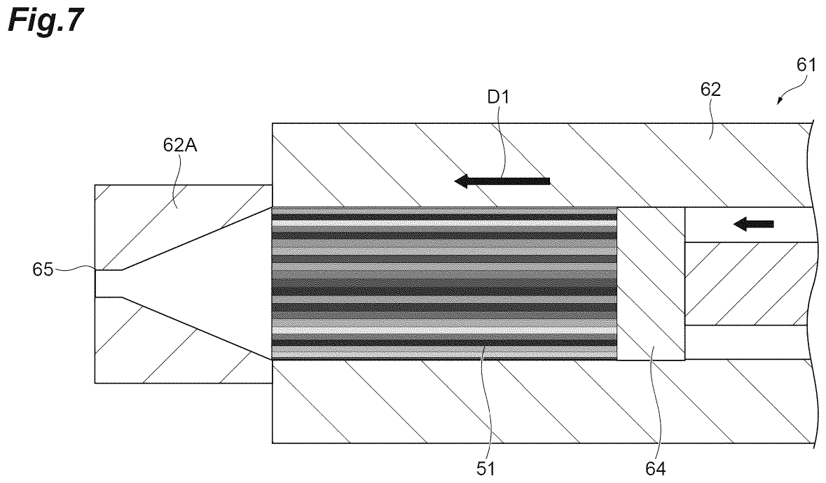

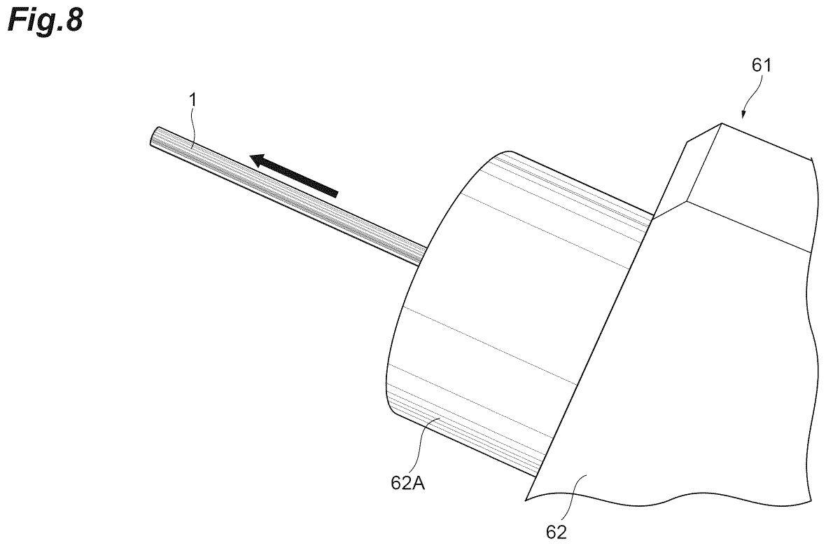

Subsequently, as illustrated in FIGS. 7 and 8, the bulk mass 51 is extrusion molded by an extrusion molding machine 61 to obtain the stick-shaped cosmetic 1 (step S4). In step S4 described above, the bulk mass 51 is supplied into a cylinder 62 of the extrusion molding machine 61 with the axial direction as a molding direction D1, and the bulk mass 51 is extruded along the molding direction D1 by a piston 64. As a result, the bulk mass 51 is stretched at a reduced diameter portion (near a nozzle 65) of a tip portion 62A of the cylinder 62, and at the same time, its outer peripheral portion moves while being rubbed against an inner wall surface, and is squeezed and extruded from the nozzle 65 as the stick-shaped cosmetic 1. As described above, for example, the stick-shaped cosmetic 1 having a diameter (width) of 1/10 of a diameter (width) of the bulk mass 51 is obtained.

As described above, in the stick-shaped cosmetic 1 according to the present embodiment, aesthetics can be enhanced by the color pattern of the multicolor region 11 of the cross section. In addition, the area ratio of the regions of each color in the multicolor region 11a (see FIG. 3A) of the first cross section is equal to the area ratio of the regions of each color in the multicolor region 11b (see FIG. 3B) of the second cross section. Therefore, it is possible to express the same application color (that is, consistent color usage) in each of a case of applying so that the respective colors of the multicolor region 11a are mixed when the first cross section is an application surface and a case of applying so that the respective colors of the multicolor region 11b are mixed when the second cross section is an application surface, and it is possible to obtain practical usability. Therefore, it is possible to obtain the stick-shaped cosmetic 1 having excellent aesthetics and usability.

In the stick-shaped cosmetic 1, the multicolor region 11 includes a pattern composed of at least one of a dot pattern, a marble pattern, a camouflage pattern, and a mosaic pattern. In this case, aesthetics can be further enhanced by the color pattern of the multicolor region 11.

In the stick-shaped cosmetic 1, the arrangement of the regions of the respective colors in the multicolor region 11a of the first cross section is different from the arrangement of the regions of the respective colors in the multicolor region 11b of the second cross section. In this case, different patterns can be included in the multicolor region 11a of the first cross section and the multicolor region 11b of the second cross section.

In the stick-shaped cosmetic 1, the outer peripheral surface of the stick-shaped cosmetic 1 includes a stripe pattern composed of a plurality of linear regions extending along the axial direction and color-coded. In this case, aesthetics can be further enhanced by the stripe pattern on the outer peripheral surface. In the stick-shaped cosmetic 1, it is possible to obtain an appearance of a multicolor pattern that differs between the cross section and the outer peripheral surface.

In the stick-shaped cosmetic 1, among the plurality of regions color-coded with the N types of colors in the multicolor region 11, the area of the widest maximum region is 10% or less of the total area of the multicolor region, and the area of the narrowest minimum region is 1% or less of the total area of the multicolor region. In this case, a fine color pattern can be included in the multicolor region 11.

In the stick-shaped cosmetic 1, the number of times that the center line passing through the center of the multicolor region 11 intersects the boundary lines of the plurality of regions color-coded with the N types of colors is five times or more. In this case, a fine color pattern can be included in the multicolor region 11.

In the stick-shaped cosmetic 1, the boundary lines of the plurality of regions color-coded with the N types of colors in the multicolor region 11 are amorphous. In this case, the color pattern composed of the plurality of regions having the amorphous outline can be included in the multicolor region 11.

The stick-shaped cosmetic 1 can express a predetermined color obtained by mixing the N types of colors as an application color. In this case, when the stick-shaped cosmetic 1 is applied so that respective colors in the multicolor region 11 are mixed, the predetermined color can be expressed as the application color.

Here, as described above, when the bulk mass 51 is extruded by the extrusion molding machine 61 in the molding direction D1, which is the axial direction, the outer peripheral surface of the bulk mass 51 is rubbed against the inner wall surface of the tip portion 62A of the extrusion molding machine 61 along the axial direction. At this time, pressure is applied in the vicinity of the nozzle 65 of the tip portion 62A of the extrusion molding machine 61, and the bulk mass 51 is stretched. Even when the bulk mass 51 is stretched as described above, in the method for producing the stick-shaped cosmetic 1 according to the present embodiment, the core materials included therein are stretched in the axial direction, and it is possible to suppress mixing of the plurality of different core materials, and thus it is possible to suppress mixing of the plurality of types of colors. Therefore, in the produced stick-shaped cosmetic 1, it is possible to suppress the plurality of types of colors from becoming dirty due to mixing on the outer peripheral surface, and to enhance aesthetics. On the outer peripheral surface of the stick-shaped cosmetic 1, it is possible to prevent the boundaries of the colors from being unclear and to form elongated vivid color streaks.

In the method for producing the stick-shaped cosmetic 1, the plurality of core materials (a plurality of core materials packed in the cylindrical member) of the N types of colors configuring the bulk mass 51 may be randomly arranged. In this case, the color pattern of the multicolor region 11 can be randomly configured.

In the method for producing the stick-shaped cosmetic 1, the plurality of core materials of the N types of colors configuring the bulk mass 51 are arranged so as to form a pattern composed of at least one of a dot pattern, a marble pattern, a camouflage pattern, and a mosaic pattern when viewed from the axial direction. In this case, in the produced stick-shaped cosmetic 1, aesthetics can be enhanced by the color pattern included in the multicolor region 11.

The method for producing the stick-shaped cosmetic 1 includes a step of bundling the plurality of core materials of the N types of colors while mixing them with the axial direction aligned, and packing the plurality of core materials into the cylindrical member, and a step of forming the bulk mass 51 by compressing and integrating the plurality of core materials packed in the cylindrical member in the axial direction. In this case, molding by the extrusion molding machine 61 can be easily and reliably performed.

In the method for producing the stick-shaped cosmetic 1, the maximum width of the cross section of the core material is 0.1 to 10 mm. By using such a thin core material, a fine color pattern can be included in the multicolor region 11 of the produced stick-shaped cosmetic 1.

In the method for producing the stick-shaped cosmetic 1, the oily component of the core material is 30 mass % or more and 80 mass % or less. In this case, since the bulk mass 51 is easily stretched when rubbed against the inner wall surface of the extrusion molding machine 61, the above effect of suppressing mixing of the plurality of types of colors is particularly effective.

In the method for producing the stick-shaped cosmetic 1, the bulk mass 51 may include 12 or more core materials. In this case, various color patterns can be included in the multicolor region 11 of the produced stick-shaped cosmetic 1. In the above, step S1 described above corresponds to a first step. The steps S2 to S4 described above correspond to a second step.

Although the embodiment of the stick-shaped cosmetic and the method for producing the same according to the present disclosure have been described above, the stick-shaped cosmetic and the method for producing the same according to the present disclosure are not limited to the contents of the above-described embodiment.

In the above-described embodiment, for example, the number of core materials used, the cross-sectional shape of the stick-shaped cosmetic 1, the shape of the pattern of the multicolor region 11, the color arrangement pattern of the pattern of the multicolor region 11, the denseness of the pattern of the multicolor region 11, and the like are not particularly limited.

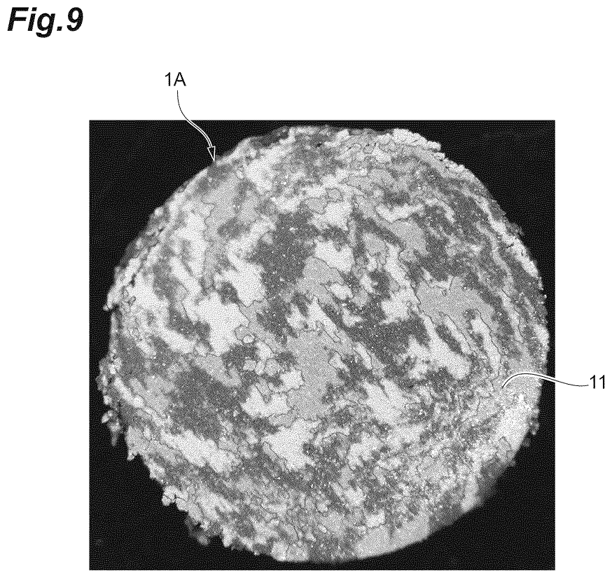

As an example, a total of 450 core materials may be used, 150 core materials for each of the N types of colors. In this case, a stick-shaped cosmetic 1A illustrated in FIG. 9 can be obtained. The stick-shaped cosmetic 1A has a circular cross section. The area of each region in the multicolor region 11 of the stick-shaped cosmetic 1A may be 0.2% to 5% of the total area of the multicolor region 11. The number of times that the center line passing through the center of the multicolor region 11 of the stick-shaped cosmetic 1A intersects the boundary lines of the plurality of regions may be 30 times or more.

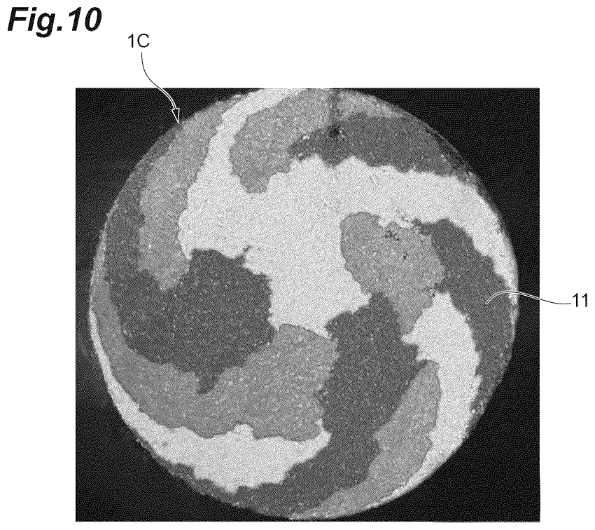

As another example, a total of 18 core materials may be used, six core materials for each of the N types of colors. In this case, a stick-shaped cosmetic 1C illustrated in FIG. 10 can be obtained. The stick-shaped cosmetic 1C has a circular cross section. The area of each region in the multicolor region 11 of the stick-shaped cosmetic 1C may be 5.3% to 23.2% of the total area of the multicolor region 11. The number of times that the center line passing through the center of the multicolor region 11 of the stick-shaped cosmetic 1C intersects the boundary lines of the plurality of regions may be five times or more.

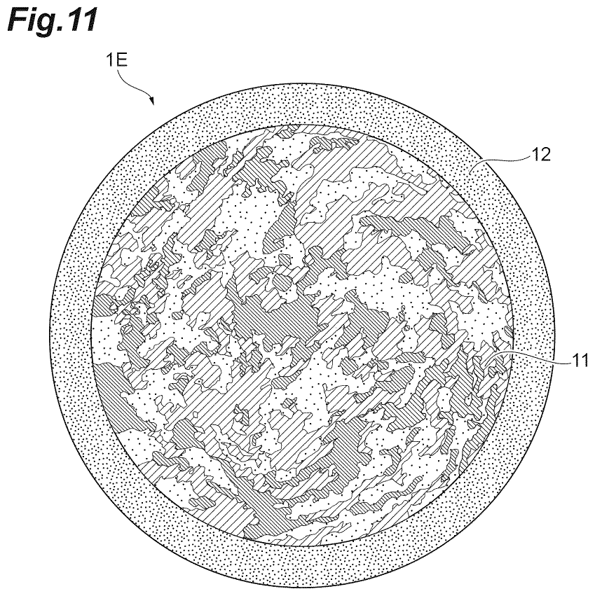

The stick-shaped cosmetic of the above-described embodiment may have a region of a predetermined color, which is an application color obtained by mixing the N types of colors, on the outer peripheral surface (side surface). For example, in a stick-shaped cosmetic 1E illustrated in FIG. 11, the region of the predetermined color may be provided at the outer edge portion of the cross section, and the entire outer peripheral surface of the stick-shaped cosmetic 1E may be the region of the predetermined color. In such a stick-shaped cosmetic 1E, the predetermined color as the application color can appear on the appearance. Therefore, a user can easily grasp the application color. For example, in the stick-shaped cosmetic 1E illustrated in FIG. 11, the region of the predetermined color may be provided in a part of the outer edge portion of the cross section, and a part of the outer peripheral surface of the stick-shaped cosmetic 1E may be the region of the predetermined color.

In the above-described embodiment, a core material having a polygonal cross-sectional shape may be used. In this case, a core material having a polygonal cross-sectional shape can be used. The cross-sectional shape of the core material is not particularly limited, and may be an elliptical shape.

In the above-described embodiment, the bulk mass 51 is formed as the intermediate body, but the bulk mass 51 may not be formed (steps S2 and S3 described above are not performed), and the plurality of core materials of the N types of colors may be bundled with the axial direction aligned. In this case, a guide having an arbitrary shape may be inserted into the cylinder 62 of the extrusion molding machine 61, and the plurality of core materials may be arranged therein. In the above-described embodiment, powder or paste may be applied to the core material, or powder or paste may be applied to the bundle of the plurality of core materials. In the above-described embodiment, an oil absorption amount or hardness of the core material is not limited, and may be various oil absorption amounts or hardness.

In the above-described embodiment, 0.1 mm as a value of the maximum width of the cross section of the core material may be approximately 0.1 mm, and may have an error in measurement or the like. In the above-described embodiment, 10 mm as the value of the maximum width of the cross section of the core material may be approximately 10 mm, and may have an error in measurement or the like. In the above-described embodiment, 30 mass % as the oily component of the core material may be approximately 30 mass %, and may have an error in measurement or the like. In the above-described embodiment, 80 mass % as the oily component of the core material may be approximately 80 mass %, and may have an error in measurement or the like.

In the above-described embodiment, the term “equal” may be not only completely equal but also substantially equal, and allows a difference in a degree of an error in measurement or the like. In the above-described embodiment, 10% of the total area of the multicolor region 11 may be approximately 10%, and may have an error in measurement or the like. In the above-described embodiment, 1% of the total area of the multicolor region 11 may be approximately 1%, and may have an error in measurement or the like.

Each configuration in the above-described embodiment or modification can be arbitrarily applied to each configuration in another embodiment or modification. Apart of each configuration in the above-described embodiment or modification can be appropriately omitted without departing from the gist of one aspect of the present invention. The shape, position, size, number, material, and arrangement mode of each portion of the above-described embodiment or modification can be appropriately changed within the scope of the above-described gist.

According to one aspect of the present disclosure, it is possible to provide a stick-shaped cosmetic having excellent aesthetics and usability. Another aspect of the present disclosure can provide a method for producing a stick-shaped cosmetic capable of enhancing aesthetics.

What is claimed is:

1. A stick-shaped cosmetic of which a cross section perpendicular to an axial direction has a multicolor region including a plurality of regions color-coded with N types of colors (N is an integer of 2 or more), wherein
the cross section includes a first cross section and a second cross section different from the first cross section, and
an area ratio of the regions of each color in the multicolor region of the first cross section is equal to an area ratio of the regions of each color in the multicolor region of the second cross section.

2. The stick-shaped cosmetic according to claim 1, wherein the multicolor region includes a pattern composed of at least one of a dot pattern, a marble pattern, a camouflage pattern, and a mosaic pattern.

3. The stick-shaped cosmetic according to claim 1, wherein an arrangement of the regions of the respective colors in the multicolor region of the first cross section is different from an arrangement of the regions of the respective colors in the multicolor region of the second cross section.

4. The stick-shaped cosmetic according to claim 1, wherein a side surface of the stick-shaped cosmetic includes a stripe pattern composed of a plurality of linear regions extending along the axial direction and color-coded.

5. The stick-shaped cosmetic according to claim 1, wherein among the plurality of regions color-coded with the N types of colors in the multicolor region, an area of a widest maximum region is 30% or less of a total area of the multicolor region, and an area of a narrowest minimum region is 0.1% or more of the total area of the multicolor region.

6. The stick-shaped cosmetic according to claim 1, wherein a number of times that a center line passing through a center of the multicolor region intersects boundary lines of the plurality of regions color-coded with the N types of colors is five times or more.

7. The stick-shaped cosmetic according to claim 1, wherein boundary lines of the plurality of regions color-coded with the N types of colors in the multicolor region are amorphous.

8. The stick-shaped cosmetic according to claim 1, wherein a predetermined color obtained by mixing the N types of colors is expressed as an application color.

9. A method for producing a stick-shaped cosmetic of which a cross section perpendicular to an axial direction has a multicolor region including a plurality of regions color-coded with N types of colors (N is an integer of 2 or more), the method comprising:
a first step of preparing a plurality of monochromatic core materials configuring the stick-shaped cosmetic for each of the N types of colors; and
a second step of, after the first step, extruding the plurality of the core materials of the N types of colors with the axial direction aligned or an intermediate body including the plurality of the core materials of the N types of colors along the axial direction by a molding machine to obtain the stick-shaped cosmetic.

10. The method for producing a stick-shaped cosmetic according to claim 9, wherein in the second step, the plurality of core materials of the N types of colors in the intermediate body are randomly arranged.

11. The method for producing a stick-shaped cosmetic according to claim 9, wherein in the second step, the plurality of core materials of the N types of colors in the intermediate body are arranged so as to form a pattern composed of at least one of a dot pattern, a marble pattern, a camouflage pattern, and a mosaic pattern when viewed from the axial direction.

12. The method for producing a stick-shaped cosmetic according to claim 9, wherein
the second step includes
a step of bundling the plurality of core materials of the N types of colors while mixing them with the axial direction aligned, and packing the plurality of core materials into a cylindrical member, and
a step of forming a columnar intermediate body by compressing and integrating the plurality of core materials packed in the cylindrical member along the axial direction.

13. The method for producing a stick-shaped cosmetic according to claim 9, wherein a cross-sectional shape of the core material is a polygonal shape.

14. The method for producing a stick-shaped cosmetic according to claim 9, wherein a maximum width of the cross section of the core material is 0.1 to 10 mm.

15. The method for producing a stick-shaped cosmetic according to claim 9, wherein an oily component of the core material is 30 mass % or more and 80 mass % or less.

16. The method for producing a stick-shaped cosmetic according to claim 9, wherein the intermediate body includes 12 or more core materials.
